(19) United States
(12) Patent Application Publication
(10) Pub. No.: US 2020/0173917 A1
(43) Pub. Date: Jun. 4, 2020

(54) DEVICE FOR HANDLING AND/OR MACHINING A WORKPIECE, AND METHOD

(71) Applicant: HOMAG Bohrsysteme GmbH, Herzebrock-Clarholz (DE)

(72) Inventor: Thomas BETTERMANN, Bielefeld (DE)

(21) Appl. No.: 16/640,719

(22) PCT Filed: Aug. 29, 2018
(86) PCT No.: PCT/EP2018/073196

§ 371 Date: Feb. 20, 2020

(30) Foreign Application Priority Data
Aug. 30, 2017 (DE) 10 2017 119 898.9

(51) Int. Cl. G01N 21/47; G01N 21/3581; G01N 33/46; G01N 21/84; G01N 22/00; G06K 1/12

(52) U.S. Cl. CPC G01N 21/47; G01N 21/3581; G01N 33/46; G01N 21/84; G01N 2021/845; G06K 1/128; G01N 2021/4735; G01N 2021/4709; G01N 22/00

(57) ABSTRACT

A device (10, 100) for handling and/or machining a workpiece (1) having a non-metal surface, comprising: a workpiece identification device (20) which is designed to irradiate electromagnetic radiation inside the workpiece (1) and to receive electromagnetic radiation reflected from the inside of the workpiece (1); and a data processing unit (30) which is configured to determine information from the inside of the workpiece (1) on the basis of measured data of the reflected electromagnetic radiation. Also disclosed is a method with which it is possible to determine a characteristic material parameter, preferably a component-specific reflection value of the workpiece (1), on the basis of the received electromagnetic radiation.

### TECHNICAL FIELD

The invention relates to a device for handling and/or machining a workpiece, comprising a workpiece identification device.

The workpieces to be machined within the context of the present invention are components used in the furniture and structural elements industry such as solid wood boards or chipboards, MDF panels, composite material workpieces and the like.

### PRIOR ART

Devices for machining a workpiece from the field of the furniture and structural elements industry are continually subject to new and increasingly stringent requirements with respect to machining quality and speed. An accurate and quick identification of a workpiece is of great importance for the machining thereof and the resulting productivity of the machine, particularly when setting machining parameters for static processing machines, i.e. machines with which the workpiece is firmly clamped and a machining head moves over the workpiece, and for conveying processing machines, i.e. machines with which the workpiece is moved during the machining thereof.

Until now, known embodiments of devices for machining workpieces comprising workpiece identification devices have determined information for identifying a workpiece by means of markings, stickers, labels or special chips, for example. This is disadvantageous since when machining a marked surface, the marking disappears and therefore the workpiece information is lost. Thus, when labeling, the respective machining steps of the corresponding surfaces must always be taken into consideration, which slows down the marking and machining processes considerably and reduces productivity.

### SUBJECT MATTER OF THE INVENTION

An object of the present invention is to provide a device and a method with which the aforementioned problems can be resolved at least in part, and/or with which a secure information base for workpieces to be machined can be created.

According to the invention, this object is achieved by a device having the features of claim 1, and by a method according to claim 12. Further preferred embodiments can be found in the dependent claims.

According to the invention, a device for handling and/or machining a workpiece having a non-metallic surface comprises: a workpiece identification device which is designed to irradiate electromagnetic radiation inside the workpiece and to receive electromagnetic radiation reflected from the inside of the workpiece, and a data processing unit which is configured to determine information from the inside of the workpiece on the basis of measured data of the reflected electromagnetic radiation.

Furthermore, the device for machining a workpiece can comprise a machining device. The machining device is designed to machine a workpiece on the basis of the information from the data processing unit.

One example of a workpiece typically used is a clamping plate having coating material on the main sides thereof, as is customary in the case of kitchen worktops.

By irradiating electromagnetic radiation inside the workpiece, it is possible to obtain information regarding, for example, the density of a solid wood board, i.e. a characteristic material parameter of the workpiece, and as a result to optimally set machining parameters, such as the feed rate of a machining saw, and to identify material parameters, such as the type of wood. This has the advantage of improved machining, which results in an increased machining speed and also higher quality machining. By implication, this also means for the device that an overload thereof can be prevented and therefore the service life of the device can be extended. This also has a positive effect on the service life of the machining tools such as drill heads, sanding belts etc., and therefore operating costs are reduced.

Furthermore, as a result of the irradiation of electromagnetic radiation inside the workpiece and the reflection thereof it is possible to identify more accurately the type of the workpiece with the help of the material data determined by the data processing unit.

In addition, the characteristic material parameter determined by the data processing unit in conjunction with the workpiece identification device can contribute to the identification of the workpiece.

In this way, components can be clearly identified and machined regardless of changes to the shape, color or surfaces thereof, without the need to consider the respective machining steps of the workpiece. Moreover, as a result of identifying the component through the workpiece interior it is possible to machine the component from all sides in an optimum manner, and disfiguring the workpiece by having to make marks is unnecessary.

It is also preferred that the internal structure of the workpiece is reproduced or retrieved in a line or plane that is perpendicular to a workpiece surface and/or in a line or plane that is parallel to a workpiece surface.

With such an embodiment, it is possible for the depth of the workpiece to be analyzed through a vertical profile through the workpiece and therefore any imperfections, knots, gaps and the like can be detected. In the case of an analysis of the workpiece parallel to the workpiece surface, an analysis of the workpiece interior can be performed over the entire length or width of the workpiece.

According to a further embodiment, the device described above comprises a conveying device. This conveying device moves a workpiece at a speed, wherein it is preferred that the speed is set on the basis of the information determined by the data processing unit.

In this way, it is possible to determine using the data obtained from the data processing unit optimal machining parameters, adapted to the workpiece and the internal profile thereof. One example of an important machining parameter is the speed of movement of a machining head. It would also be possible, for example, to set a conveying speed of the workpiece relative to the machining head. A further field of application is the detection of imperfections, such as knotholes, or inhomogeneity/material defects in a solid wood board.

According to a further aspect of the invention, the machining device comprises a machining device for machining a workpiece, wherein the machining device is configured to machine the workpiece on the basis of the information determined by the data processing unit or wherein on the basis of the information determined by the data processing unit the machining device is activated or the operation thereof is changed.

As a result of such an aspect it is possible to control the machining explicitly using the information from the data processing unit and/or the workpiece identification device and to thereby control the machining device.

According to a further aspect of the disclosure, the electromagnetic radiation of the sensor arrangement or the workpiece identification device is based on radar radiation or terahertz radiation.

This has the advantage that a broad frequency spectrum of radiation can be used. Furthermore, radar radiation is particularly suitable since the frequency range of the radiation is not in the visible range of light for humans, and therefore there is no need for special considerations in terms of machining or determining material parameters using radar radiation when machining. Thus, workpiece identification with cameras, for example, is not limited by a lack of lighting. In contrast to workpiece identification devices with camera systems, factories do not have to be darkened, or the lighting thereof does not have to be adjusted. Thus, components can be clearly identified and machined regardless of changes to the shape, color or surfaces thereof in a way that is not harmful to workers and irrespective of the surrounding conditions in the factory.

According to a further aspect of the preferred embodiment, the radar radiation is outside the visible spectral range, typically between 70 and 75 GHz.

In this range radar radiation has a short wavelength and is therefore well suited for identifying material properties in a processing machine. Typically, the so-called W band is used here. With the frequency range of between 70 and 75 GHz properties of the workpiece interior can be determined up to a distance of a few meters. Thus, the shape of a workpiece can be freely selected since the workpiece identification device together with the enclosed sensor arrangement can be attached to the workpiece at a sufficient distance therefrom and therefore, for example, the machining or identification of larger components is not further restricted.

However, measuring in close proximity to the workpiece is also achievable. Disturbance variables as a result of the reflection of particles in the air can therefore be prevented.

According to a further embodiment of the invention, the electromagnetic radiation of the sensor arrangement is based on microwave radiation.

Microwave radiation makes is possible to provide a more favorable embodiment of such a device and also facilitates a more specific measurement range of frequencies, such that this radiation is not in the visible spectral range. This is advantageous since the machining of a workpiece can therefore be carried out irrespective of the conditions of the surrounding light. Thus, in contrast to workpiece identification devices with camera systems, as already described above, it is not necessary to darken factories or adjust the lighting thereof. Components can therefore be clearly identified and machined regardless of changes to the shape, color or surfaces thereof. Moreover, adverse effects for workers are thereby excluded and safety areas do not have to be observed.

Typically, this microwave radiation is in a frequency range of between 1 and 300 GHz. In this range, the operator of the machine is not adversely affected and the machining of the workpiece can be improved using the information determined with the help of the microwave radiation. Thus, in contrast to workpiece identification devices with camera systems, it is not necessary to darken factories or adjust the lighting thereof.

In a preferred embodiment, the device is configured to determine the external structure of the workpiece.

By irradiating electromagnetic radiation into the workpiece interior, properties of the workpiece can be determined possibly by using a characteristic material parameter for identifying a workpiece. These properties of the external structure serve to improve machining, with machining of a higher quality and at a higher speed consequently taking place. Thus, the productivity of the device can be increased. Moreover, by irradiating the workpiece with electromagnetic radiation along an entire direction, the external shape thereof can be determined at any point during the machining steps. It is therefore possible to provide information regarding the progress of the machining as well as information regarding the external appearance of the workpiece as early as during the machining thereof. As a result, productivity can be further increased and more information can be provided regarding the workpiece which is to be or which has been machined.

According to a further embodiment, it is possible to arrange the sensor arrangement at more than one location in the workpiece identification device so as to be able to determine properties regarding the profile of the material.

Thus, through the profile of the workpiece, information can be continuously determined from the interior of the workpiece, particularly in the case of very long workpieces which are continuously machined. It is possible with a characteristic material parameter to respond to a change to the workpiece as early as during the machining thereof, and also to optimize machining. As a result, the speed of machining is increased and the identification of a component is facilitated.

According to a further aspect of the present invention, the device comprises a marking device with which it is possible to introduce microwave markings inside the workpiece or to make a mark on the workpiece using a laser. If, for example, a mark is introduced using a laser on a workpiece surface or a region of the workpiece close to the surface thereof, this can subsequently be coated (e.g. lacquer, film, paper etc.). The material change introduced by the laser can be detected by the workpiece identification device described above and can be evaluated accordingly.

In one specific application, a plurality of markings can therefore be introduced into the original workpiece for example using a laser, such that partial workpieces later separated off can be attributed to the original workpiece.

In this way it is possible to change the internal structure of the component in a targeted manner and thereby facilitate the identification of the workpiece. As a result it is possible to save having to apply marks, markings, stamps or other means of identification. It may even be that this is not expedient in any case since it is possible that as a result of a plurality of consecutive machining steps all of the surfaces of the workpiece are machined and the markings are lost. However, if the markings are introduced inside the workpiece by the marking device, it is possible to retain permanent and individual information regarding the component for the entire machining step or the entire machining process and beyond, without tarnishing the exterior of the workpiece or having to take the markings into consideration during machining. This increases productivity and prevents the production of defective goods. Thus, components can be clearly identified regardless of changes to the shape, color or surfaces thereof and defective products can be avoided.

It is also preferred that the marking device is placed in a feed point of the device, at which the workpiece is fed to the device and wherein the information determined by the data processing unit is based on the markings of the marking device.

With such an embodiment it is possible that the workpiece can be provided with a marking prior to machining and therefore it can be provided with an identification, for example, even before the start of the machining step(s).

The present invention further comprises a method for evaluating a workpiece, having the following steps: emitting electromagnetic radiation in the direction of the workpiece; receiving the electromagnetic radiation reflected from the inside of the workpiece; and determining a characteristic material parameter, preferably a component-specific reflection value of the workpiece, on the basis of the received electromagnetic radiation.

One example of a workpiece typically used is a clamping plate having coating material on the main sides thereof, as is customary in the case of kitchen worktops. Another example is a solid wood board.

Using this method it is possible to detect the interior of a workpiece having a non-metal surface, so as to be able to thereby adapt the machining steps to the properties of the workpiece. With this method it is possible to ensure quicker and higher quality processing of the workpiece. Moreover, as a result of the information determined regarding the component itself, for example, the function of the finished product, the production stage, the completion date and the necessary machining steps for this component, relevant data can be automatically provided individually and quickly without changing the exterior of the workpiece.

In a further step of the method, it is possible by way of microwaves or ultrasound, for example, to introduce information into the workpiece or to provide this information by means of laser radiation on the workpiece, for example on the surface of the workpiece or on a region of the workpiece close to the surface thereof. The workpiece can subsequently be coated.

The internal structure of the workpiece can thereby be changed in a targeted manner and as a consequence the identification of the workpiece is facilitated. Thus, regardless of changes to the shape, color or surfaces thereof, the workpieces can be clearly identified and above all labeled throughout the machining process thereof. Labeling in the traditional sense is therefore no longer necessary. The machining process is thereby accelerated and the number of defective goods produced is reduced. By introducing markings inside the workpiece, manual identifications can be avoided and therefore productivity is increased further and the number of erroneous designations is reduced as a result of automating the labeling step during the machining step.

In a further step of the method, information introduced by means of the marking device can be read out in an additional step.

In this way it is possible to determine information regarding the respective machining state, the workpiece itself, the condition of said workpiece or project-related information as early as during machining.

In a further aspect of the method it is possible to move the workpiece and the workpiece identification device relative to one another for the measurement. Thus, successive measurement values can be detected for a plurality of measuring points in the workpiece interior without moving the workpiece identification device, and a characteristic material parameter can be determined for the properties of the workpiece on the basis of these measurement values.

This has the advantage that a measurement can be ascertained without stopping the workpiece, i.e. while it is being transferred to the next machining step, or even during the machining step without stopping the system. In this way, the speed of the process can be increased and general productivity is also increased.

According to a further aspect of the method it is possible to raise the intensity of the radar radiation. As a result, successive measurement values are detected for a plurality of measuring sites at different depths of the workpiece interior. It is therefore possible to determine a characteristic material parameter for the properties of the workpiece on the basis of the measurement values.

As a consequence, an all-round image of the workpiece can be determined through a depth profile in the cross-section of the workpiece and/or through the profile of the width and/or length of the workpiece. Thus, inhomogeneity, defects or imperfections as a result of knots in the workpiece, for example, can be detected early, poor quality of the material can be ruled out and optimized machining can be ensured. Alternatively, it is possible to change the intensity by changing the focus of the radiation.

According to a further aspect of the method, it is possible to clearly identify the workpiece using the characteristic material parameter and as a result information can be relayed on via various machines to the next machining step.

This makes it possible to continually read out the information throughout the entire machining process with a plurality of individual machining steps, with the help of the information from inside the workpiece and therefore to clearly identify the workpiece at any point in the process without the need for contact. In this way, components can be clearly identified regardless of changes to the shape, color or surfaces thereof.

A further application is, for example, the coding of a workpiece, wherein the coding may contain data regarding, for example, the type of machining, the origin, the machining site, the machine type etc. Furthermore, it is possible that by clearly labeling the component the processing machines are able to store information regarding the workpiece in the “cloud” and the current machining state thereof, for example, can be retrieved from anywhere in the world. The term “cloud” is to be understood here to be the decentralized storage of data on an external server or storage medium, which can be accessed from anywhere in the world via a long-distance communications link, in particular in the form of the internet or an intranet.

In a further step of the method, the characteristic material parameter serves to identify the material and as a result information can be relayed on to the next machining step.

With such an embodiment it is possible to optimize machining using the parameters determined.

In a further aspect of the method it is possible to carry out the method with the device described above.

The aforementioned method steps can be stored in a data processing unit of the previously described device for machining a workpiece. The data processing unit can therefore be configured to carry out these steps.

According to a further objective, a marking device configured to introduce information into the workpiece, in particular by way of microwave radiation or ultrasound, is provided. The marking device can be combined with one of the aforementioned aspects or with one of the dependent claims.

These markings are introduced by changing the internal structure of the workpiece with the help of radiation, preferably microwave radiation, terahertz radiation or even ultrasound. To this end the marking device has a marking unit which preferably directs the radiation to the interior of the workpiece.

According to yet another objective, the marking device is one which can introduce a marking onto a workpiece surface or a region close to the surface of the workpiece using a laser. Subsequently, the surface of the workpiece is coated, for example (e.g. lacquer, film, paper etc.). The material change introduced by the laser can for example be detected by the workpiece identification device described above and can be evaluated accordingly.

As a consequence of the absorption of the electromagnetic radiation inside the workpiece, there may be localized heating, wherein chemical conversion processes or combustion processes, facilitated by absorbent particles for example, bring about a permanent structural change inside the workpiece. The marking introduced accordingly is not visible from the outside. The information introduced can for example be used to clearly mark the workpiece.

### BRIEF DESCRIPTION OF THE DRAWINGS

In the following, embodiment examples of the invention will be described in more detail with the help of the enclosed drawings.

FIG. 1: Schematic illustration of a device for machining a workpiece having a non-metal surface, comprising a workpiece identification device according to a first embodiment.

FIG. 2: Schematic illustration of a device for machining a workpiece having a non-metal surface, comprising a workpiece identification device and a marking device according to a further embodiment.

FIG. 3: Schematic illustration of the introduction of a marking into the workpiece and the result of the workpiece identification device reading out the marking.

FIG. 4: Schematic illustration of a further embodiment in the form of a machining device comprising a marking device.

### DETAILED DESCRIPTION OF THE EMBODIMENTS

In the following, a device for machining a workpiece having a non-metal surface will be described with the help of the schematic drawings. General examples of such devices, also known as processing machines, include CNC machining centers, through-feed machines (for example for machining the edges of plate-shaped workpieces), sanding machines and the like. Further modifications cited in this context of certain individual features can each individually be combined in order to show new embodiments.

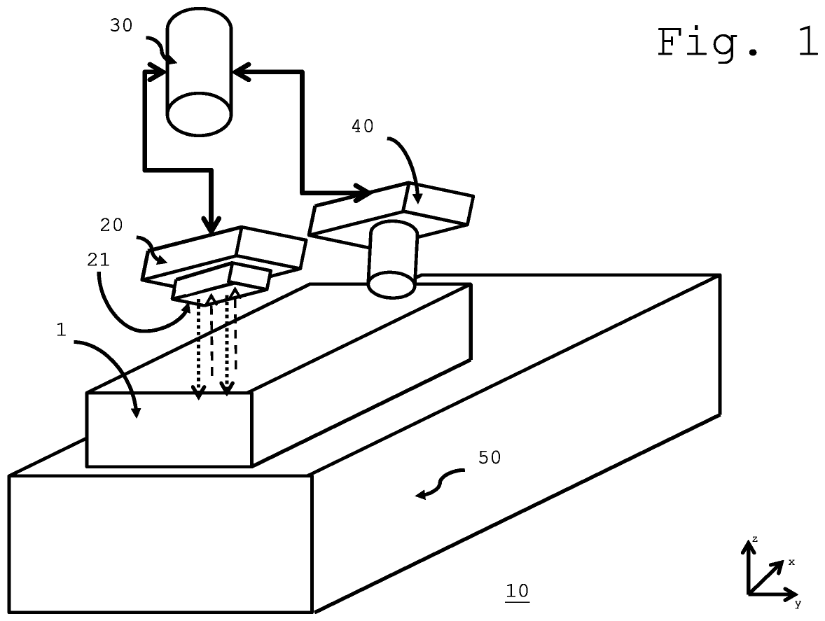

FIG. 1 schematically shows a device 10 according to a first embodiment (hereinafter referred to as the processing machine), which is suitable for machining a workpiece 1 and which comprises a workpiece identification device 20 having a sensor arrangement 21. Moreover, the processing machine 10 comprises a data processing unit 30 for determining together with the sensor arrangement 21 a characteristic material parameter of a workpiece 1. The processing machine 10 also comprises a machining device 40 for machining the workpiece. The processing machine 10 comprises a machining table 50. Alternatively, instead of a machining table 50 a support system for holding a workpiece can be provided, or one or more conveying devices.

A workpiece 1 can be positioned on the machining table 50 during the machining thereof in the processing machine 10. Furthermore, in the case of static machining, i.e. a machining process in which the workpiece is firmly clamped and a machining head of the machining device 40 moves relative thereto, it is possible to fix the workpiece 1 onto the machining table 50 by way of a fixing (not shown). As a result, it is possible to ensure an exact positioning of the workpiece 1.

In a further embodiment it is possible that for through-feed machining, i.e. a machining process in which the workpiece 1 is moved relative to the machining head of the machining device 40, the machining table 50 is moved by a conveying device. This makes it possible for the workpiece 1 to be moved with an exact position and speed by way of a conveying device. In this case the workpiece 1 is moved with a conveying device by way of stops provided on the machining table 50 and at the same time it is held in position so that it is possible for all-round machining to run smoothly.

The workpiece 1 is preferably a wooden workpiece, a plastics material workpiece or a workpiece of a comparable material that is suitable for being machined with the device 10. In addition, the workpiece 1 has a non-metal surface. There are no limitations for the geometric shape of the workpiece 1, as long as fixing can be ensured during machining.

As is described above, the device 10 comprises a workpiece identification device 20. This workpiece identification device 20 is designed to comprise a transmitter and a sensor arrangement 21 as a receiver. Moreover, the sensor arrangement 21 is connected to a data processing unit 30 which will be described in the following. The workpiece identification device is firmly connected to the device 10. However, it is also possible to fix the workpiece identification device 20 separately from the processing machine 10 to a further element, such that a modular set-up is possible for the production method having a plurality of production steps, and not every device 10 has to comprise such a workpiece identification device 20.

An arrangement of a plurality of workpiece identification devices 20 inside a processing machine 10 is also conceivable. In this way, considerably more information regarding the progress of the machining can be generated and therefore the machining process can be optimized as a result of more generated data.

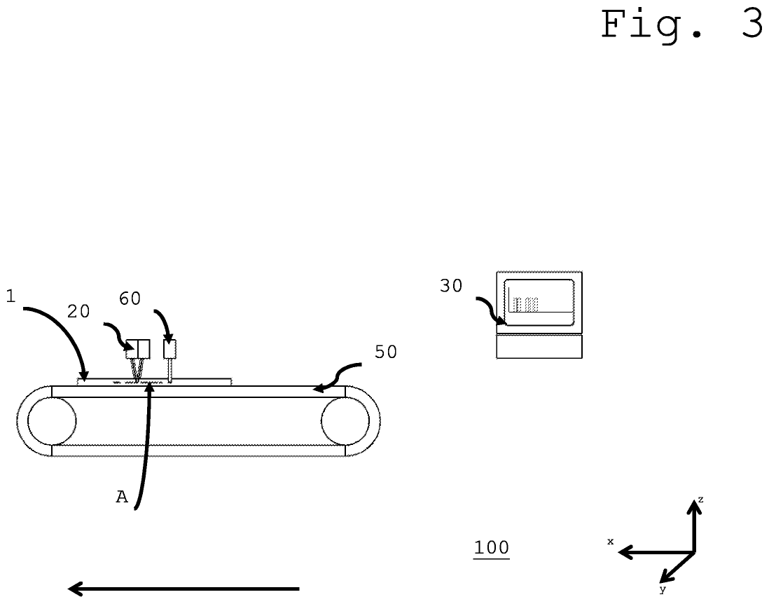

As is shown in FIG. 3, the sensor arrangement 21 of the workpiece identification device 20 is able to irradiate electromagnetic radiation inside the workpiece 1 by means of the transmitter and to receive the radiation reflected from the inside of the workpiece 1. Alternatively, the transmitted radiation can be detected. Here, the radiation can be analyzed in a scanning region (A) through the profile of the workpiece and/or at different depths within the scanning region (A) of the workpiece. The arrow pictured shows the conveying direction of the workpiece.

The distinctive feature of a wood-working machine is above all that, in contrast to metal workpieces, it is actually possible to irradiate inside the workpiece and also the reflected radiation re-emerges therefrom. From an abstract perspective, this could be compared with a passage of light through frosted glass in contrast to light irradiation in a mirror.

A transmitter is provided in the sensor arrangement 21, which transmits the aforementioned electromagnetic radiation. Moreover, a detection sensor is contained in the sensor arrangement, which can detect the electromagnetic radiation reflected from the inside of the workpiece 1.

The transmitter controls the irradiation depth into the workpiece 1 by way of the intensity of the electromagnetic radiation. Alternatively, it would also be possible to control the irradiation depth by means of a focusing device (not shown). In this way, electromagnetic radiation can be directed in a targeted manner at a specific depth of the workpiece 1. Furthermore, it is possible to determine an entire depth profile of the workpiece 1 by way of a continuous change in intensity of the electromagnetic radiation emitted by the transmitter. Further embodiments, scanning rates or scans of the workpiece interior by way of irradiating electromagnetic radiation from the transmitter 22 are conceivable.

The detection sensor detects the electromagnetic radiation reflected by the workpiece 1, which was directed by the transmitter at the workpiece interior. The degree of reflection is determined by a characteristic structure or density of the workpiece 1 and/or by the depth of penetration of the electromagnetic radiation. As a result of the characteristic structure of the workpiece, a part of the electromagnetic radiation is reflected back in the direction of the workpiece identification device or the sensor arrangement connected thereto. The data or information determined by the detection sensor (an example of this would be a layer structure or a density profile of the workpiece) is relayed on to the data processing unit 30. In an alternative embodiment, it is possible that the depth of penetration of the electromagnetic radiation is considered in the analysis.

In the case of a stationary workpiece 1, it is possible to arrange the transmitter and the detection sensor so as to be next to one another. In the case of continuous machining, the speed of movement of the workpiece 1 must be taken into account. Thus, it is necessary either to irradiate electromagnetic radiation at an angle into the workpiece 1 or to arrange the detection sensor at a sufficient distance appropriate to the speed of movement. In this regard a possible refraction of the radiation should be taken into consideration. Furthermore, it is possible to arrange the detection sensor outside of the sensor arrangement 21. In a further embodiment it is also possible to completely uncouple the sensor arrangement 21 with the transmitter from a sensor arrangement 21 with the detection sensor and therefore to divide everything into a plurality of housings.

The present embodiment describes an arrangement of one transmitter and one detection sensor. However, an embodiment of a sensor arrangement 21 having a plurality of sensors or detection sensors is conceivable.

In a further embodiment, it is possible for the workpiece identification device 20 to comprise a plurality of sensor arrangements 21. Moreover, the irradiation of an entire section (a strip) of a workpiece 1 using sensors is possible, or an irradiation by means of spaced-apart sensor arrangements 21.

The data processing unit 30 is connected to the workpiece identification device 20 or is directly connected to the sensor arrangement 21. The data determined by the detection sensor is transmitted to the data processing unit 30 via a supply line, such as an Ethernet cable for example. With the data processing unit 30 it is possible to determine information regarding the component to be machined on the basis of the data determined by the detection sensor.

Moreover, with the data processing unit 30 it is possible to transfer information for the subsequent machining of the workpiece 1 from the data of the detection sensor to the machining device 40. The data processing unit 30 is connected to the machining device 40. The information for the machining as determined by the data processing unit 30 is transmitted to the machining device via a conveying cable, such as an Ethernet cable for example. With the data transmitted by way of the conveying cable it is possible to clearly identify workpieces without the influence of external factors. Alternatively, it is also possible to introduce a label to the workpiece 1 by way of microwaves, for example, in the event that identification by means of detecting the external structure is not sufficient.

Furthermore, it is possible to adapt the machining parameters (such as the speed of rotation of a milling head or the feed rate of a machining head) of the machining device 40 using the data generated. The machining device 40 is able to machine the workpiece 1. A machining head is provided in the machining device 40. The machining head is driven by a motor and is rotatably supported by a bearing. The machining head can be moved in an X axis and a Y axis via a displacement system (not shown), such that it can be moved over a workpiece 1 and therefore over the entirety of the workpiece 1. Furthermore, the machining head is able to move in the Z axis, such that it engages with the workpiece 1 and can machine said workpiece. In the case of continuous machining it is possible that not all axes have to be designed so as to be movable, since in this case the workpiece 1 is moved or conveyed relative to the machining head. This can accelerate the production process.

In a further embodiment, it is possible that a machining head can be automatically changed during the machining process by a workpiece changing device and therefore a plurality of machining steps can be performed in the same device 10.

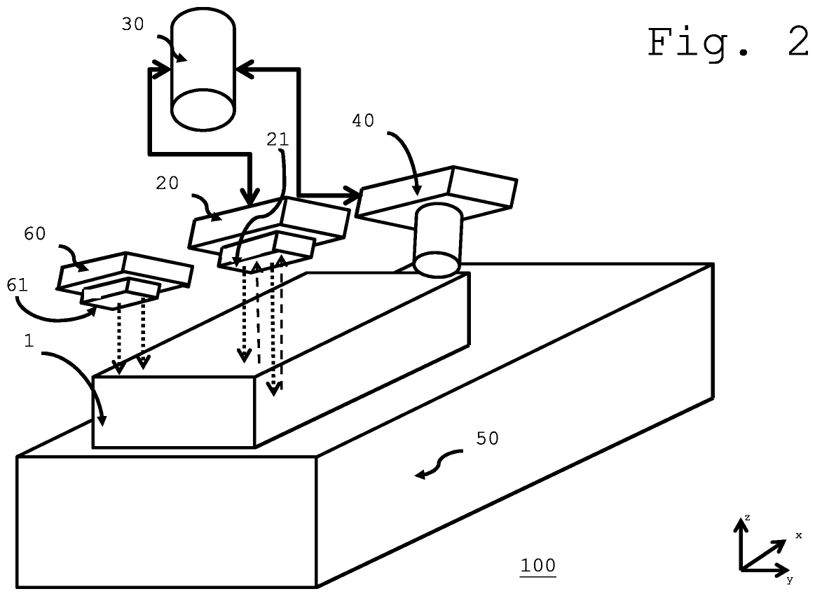

Next, a device 100 according to a further embodiment will be described with reference to FIG. 2 and FIG. 3.

In the further embodiment in FIG. 2 and FIG. 3 the same components are provided with the same reference numbers as in the first embodiment, and they will not be described in detail. The device 100 (also referred to as the processing machine) of the further embodiment is different in that a marking device 60 for introducing microwave markings inside the workpiece is provided in the device 10. The arrow pictured shows the conveying direction of the workpiece.

In this case the device 100 comprises a marking device 60 with which it is possible to introduce markings inside a workpiece prior to, during or after the machining thereof. These markings are introduced by changing the internal structure of the workpiece with the help of electromagnetic radiation, preferably microwave radiation, terahertz radiation or even ultrasound. To this end the marking device 60 has a marking unit 61, which preferably directs microwave radiation to the interior of the workpiece 1.

The use of the marking device results in localized heating as a consequence of the absorption of the electromagnetic radiation inside the workpiece, wherein chemical conversion processes or combustion processes in the wood bring about a permanent structural change inside the workpiece, which is not visible from the outside. These structural changes can be used as coding for the workpiece, in order to be able to later clearly identify the workpiece.

As a result of the change in the intensity of the microwave radiation or the adjustment of the focusing device it is also possible to make markings for identifying a workpiece at different depths of the workpiece. It is possible that the introduced markings will be read out by a workpiece identification device 20 later on in the machining process. Thus, information regarding the component can be transported. Such a step of reading out markings is shown in FIG. 3 in a schematic illustration of a data processing unit 30. In this regard damage to the workpiece surface has to be expected when laser radiation is used.

It is also possible to mark the workpiece 1 regardless of the machining steps and surface. This ensures a permanent and individual identification of the workpiece 1. Moreover, the markings are located inside the workpiece 1 and therefore they do not affect the external appearance of the workpiece 1 or the finished product resulting from the workpiece 1.

In the case of a processing machine 100, the introduction of binary information into the workpiece, for example, would be conceivable. For example, in this respect FIG. 3 shows a sequence of binary information regarding the profile of the workpiece 1 in the data processing unit 30 of FIG. 3. The arrow pictured in the lower part of the image signalizes the conveying direction of the workpiece 1. Moreover, information regarding the project, an individual product identification, the machining steps to be carried out and the client may be contained therein. In addition, information regarding the properties of the workpiece, such as the material composition or the material thickness, can be introduced. Further information can also be introduced without restrictions.

Even though a marking device 60 based on microwave radiation is preferably used within the scope of the present invention, in alternative embodiments other sensors or other physical effects for identifying workpieces and for marking workpieces can be used. In a preferred embodiment of the device, the sensor arrangement is arranged externally and is connected to the device (not shown). However, other arrangements and positions of the workpiece identification device 20 or the marking device 60 are also always conceivable.

In addition, the embodiments described have the advantage that as a result of a modular construction of the respective elements it is possible to subsequently equip further processing machines with the novel identification or marking technology as according to the present invention. In this regard only a connection to the data processing unit 30 of the processing machine 10 is necessary. Furthermore, the workpiece identification device 20 must be installed in a further device.

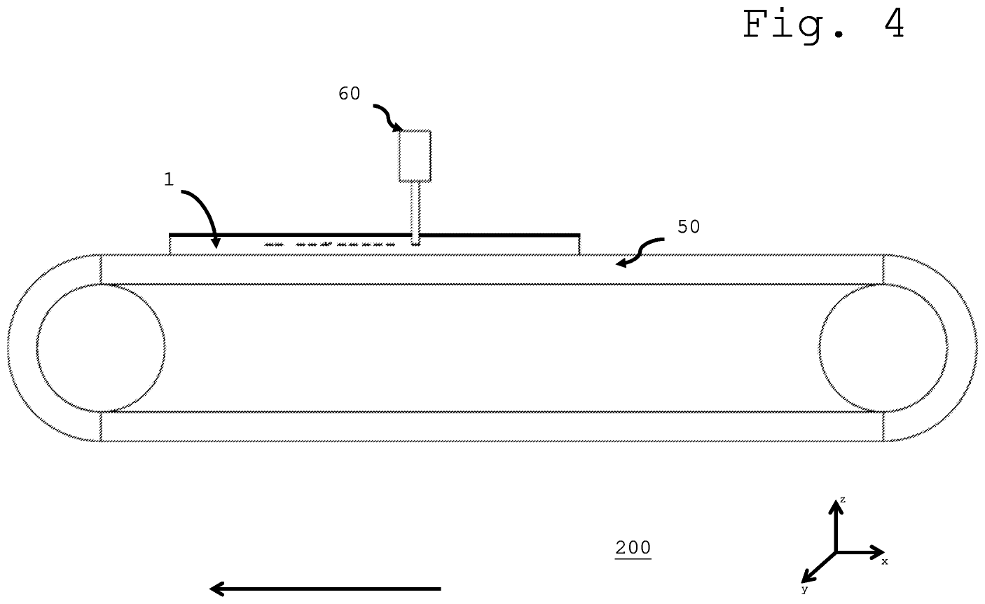

In the following, a device 200 according to a further embodiment will be described with reference to FIG. 4.

In the further embodiment in FIG. 4, the same components are provided with the same reference numbers as in the aforementioned embodiments and they will not be described in detail. The arrow pictured shows the conveying direction of the workpiece. The device 200 (also referred to as the processing machine) of the further embodiment of FIG. 4 is different in that a marking device 60 is provided in the device 10 for introducing microwave markings inside the workpiece and there is no workpiece identification device 20.

With such a device 200 it is possible to introduce markings inside the workpiece 1 using the marking device 60, and these markings can be used at a later point in time for the purpose of identification. Thus, subsequent handling and machining is made easier, since the introduced markings can be read out and therefore information is available regarding the material, the condition or the machining parameters.

The embodiments of the present invention have been described in detail with reference to the drawings, but configurations and combinations thereof in the embodiments are just examples, and additions, omissions, substitutions and other modifications thereof can be made without deviating from the scope of the present invention. Furthermore, the present invention is not restricted to these embodiments and is only limited by the scope of the enclosed claims.

According to the device and method described above it is possible to identify a workpiece by determining a characteristic material parameter from the inside of the workpiece and thereby optimize the machining thereof. It is also possible to change the interior of a workpiece in a targeted manner by way of a marking device, and therefore introduce markings regardless of changes to the shape, color or surfaces during machining.

1. A device for handling and/or machining a workpiece having a non-metal surface, comprising:
a workpiece identification device which is designed to irradiate electromagnetic radiation inside the workpiece and to receive electromagnetic radiation reflected from the inside of the workpiece, and
a data processing unit which is configured to determine information from the inside of the workpiece on the basis of measured data of the reflected electromagnetic radiation.

2. The device according to claim 1, wherein the information determined by the data processing unit depicts a characteristic internal structure of the workpiece.

3. The device according to claim 2, wherein the internal structure of the workpiece is retrieved in a line or plane that is perpendicular to a workpiece surface and/or in a line or plane that is parallel to a workpiece surface.

4. The device according to claim 1, comprising a conveying device which moves a workpiece at a speed, wherein the speed is set on the basis of the information determined by the data processing unit.

5. The device according to claim 1, further comprising a machining device for machining a workpiece, wherein the machining device is designed to machine the workpiece on the basis of the information determined by the data processing unit or wherein on the basis of the information determined by the data processing unit the machining device is activated or the operation thereof is changed.

6. The device according to claim 1, wherein the electromagnetic radiation of the workpiece identification device is in the frequency range of radar radiation or terahertz radiation.

7. The device according to claim 6, wherein the radar radiation is outside the visible spectral range, typically between 70 and 75 GHz.

8. The device according to claim 1, wherein the electromagnetic radiation of the workpiece identification device is in the range of microwave radiation, and wherein the microwave radiation is between 1 and 300 GHz.

9. The device according to claim 1, wherein the workpiece identification device is designed to determine the external structure of the workpiece.

10. The device according to claim 1, further comprising a marking device for introducing laser and/or microwave markings on the workpiece or inside the workpiece.

11. The device according to claim 10, wherein the marking device is preferably placed in a feed point of the device, at which the workpiece is fed to the device, and wherein the information determined by the data processing unit is based on the markings of the marking device.

12. A method for evaluating a workpiece,comprising:
emitting electromagnetic radiation in the direction of the workpiece;
receiving electromagnetic radiation reflected from the inside of the workpiece; and
determining a characteristic material parameter, preferably a component-specific reflection value of the workpiece, on the basis of the received electromagnetic radiation.

13. The method according to claim 12, wherein information is introduced into the workpiece by means of a marking device, in particular by way of laser radiation or microwave radiation or ultrasound.

14. The method according to claim 12, wherein information introduced by means of a marking device is read out in an additional step.

15. The method according to claim 12, wherein a workpiece and a workpiece identification device are moved relative to one another for measurement, successive measurement values are detected for a plurality of measuring points inside the workpiece and a characteristic material parameter for the properties of the interior of the workpiece is determined on the basis of the measurement values.

16. The method according to claim 12, wherein the intensity of the radar radiation is increased, as a result of which successive measurement values are detected for a plurality of measuring points at different depths of the workpiece interior and a characteristic material parameter for the properties of the workpiece is determined on the basis of the measurement values.

17. The method according to claim 12, wherein the characteristic material parameter serves to identify the material and as a result information can be relayed on to the next machining step.
